(19) United States
(12) Patent Application Publication
(10) Pub. No.: US 2018/0017497 A1
(43) Pub. Date: Jan. 18, 2018

(54) Method for measuring bismuth content in colloidal bismuth pectin or colloidal bismuth pectin-contained preparation

(71) Applicant: SHANXI ZHENDONG ANTE BIOPHARMACEUTICAL CO.,LTD., Jinzhong, Shanxi (CN)

(72) Inventors: Anping Li, Jinzhong, Shanxi (CN); Feng Cui, Jinzhong, Shanxi (CN); Ping Zhu, Jinzhong, Shanxi (CN); Zhengguo Qin, jinzhong, Shanxi (CN); Tai Zheng, Jinzhong, Shanxi (CN); Yuexla Wu, Jinzhong, Shanxi (CN)

(21) Appl. No.: 15/548,096

(22) PCT Filed: May 27, 2016
(86) PCT No.: PCT/CN2016/083739

§ 371 Date: Aug. 2, 2017

(30) Foreign Application Priority Data
Jun. 23, 2015 (CN) 201510348311.1

(51) Int. Cl. G01N 21/78; G01N 33/84

(52) U.S. Cl. CPC G01N 21/78; G01N 33/84; G01N 2400/46

(57) ABSTRACT

Provided is a method for measuring the bismuth content in a colloidal bismuth pectin or a colloidal bismuth pectin-contained preparation. A protonic acid is used to dissociate bismuth from a colloidal bismuth pectin, the principle of the characteristic reaction between bismuth and potassium iodide to produce yellow potassium bismuth iodide is used; ultraviolet-visible spectrophotometry is for measurement; an external standard method is for calculation. The method has strong specificity, a good linear relationship, high accuracy, good repeatability and high sensitivity. The method is able to act as a quality control method for bismuth in a colloidal bismuth pectin or a colloidal bismuth pectin-contained preparation to effectively control the product quality.

### CROSS REFERENCE OF RELATED APPLICATION

This is a U.S. National Stage under 35 U.S.C. 371 of the International Application PCT/CN2016/083739, filed May 27, 2016, which claims priority under 35 U.S.C. 119(a-d) to CN 201510348311.1, filed Jun. 23, 2015.

### BACKGROUND OF THE PRESENT INVENTION

### Field of Invention

The present invention relates to a method for measuring a bismuth content, and more particularly to a method for measuring a bismuth content in polymeric chelating bismuth-contained substances.

### Description of Related Arts

The colloidal bismuth pectin is a compound of uncertain constitution of the pectin and the bismuth Bi, which is yellow powder; wherein the bismuth pectin content (takes bismuth for calculation) is 14.0%-16.0%; pH is 8.5-10.5; the sedimentation rate is 1-0.97. The colloidal bismuth pectin is insoluble in ethanol, acetone, ether and other organic solvents, which is able to disperse uniformly and form stable colloidal system in the water.

The colloidal bismuth pectin substitutes small molecule acid groups with biological macromolecules pectin. Compared with other bismuth preparations such as bismuth subgallate, bismuth subnitrate, bismuth subsalicylate and bismuth potassium cirtrate, the colloidal bismuth pectin has strong colloidal characters, high viscosity and low absorption by human body. The colloidal pectin bismuth has high affinity for ulcerated mucosa, which forms chelation with the ulcer mucoprotein by bismuth to cover on the gastric mucosa. The epithelial tissues are stimulated to discharge mucus and the pepsin activity is inhibited to protect the gastric mucosa. The bismuth is able to kill the Helicobacter pylori. Compared to the conventional medicine, the colloidal bismuth pectin has strong protection for mucosa, which has been widely applied in the treatment of intestinal tract disease including peptic ulcer disease, chronic gastritis and etc. The colloidal bismuth pectin and the capsule preparation is an original ground medicine manufactured by the Taiyuan Red Star Pharmacy Plant which is the predecessor of Shanxi Zhendong Ante Biopharmaceutical Co., Ltd. The medicine gets the new drug certification and the drug production license from the Ministry of Health of the People's Republic of China in 1992 and now is listed in the Pharmacopoeia of the People's Republic of China, 2^{nd} volume of the 2010 version. The method for measuring the colloidal bismuth pectin content is same with the method for the bismuth subnitrate, bismuth subsalicylate, bismuth potassium cirtrate and etc., which is to dissolve the test article with nitric acid by heating, indicate with xylenol orange indicator, adopt complexometric titration, titrate the solution with the EDTA-2Na (ethylenediaminetetraacetic acid disodium salt dehydrate) volumetric solution until the solution shows yellow. Due to the biomacromolecule character of the colloidal bismuth pectin, an opaque turbid colloidal system tends to form in the measuring process; the xylenol orange is used for both the complexometric indicator and acid-base indicator; the color change of the indicator tends to be disturbed and is hard to be keenly observed, which effects the end point detection and compromises the repeatability and accuracy of the measurement method.

Ge xiaoming (Determination of Bismuth in Compound Tetracycline Hydrochloride Capsules by Spectrophotometry, Chinese Journal of Pharmaceutical Analysis, 2005, 25(6), 670-672) adopted ultraviolet-visible spectrophotometry to measure the bismuth salts content in the compound tetracycline hydrochloride capsule. The adopted reference article of bismuth potassium cirtrate is difficult to accurately valuate, which is not able to satisfy the accuracy requirement of the medicine content measurement method. The bismuth potassium cirtrate is a small molecule bismuth-contained compound and easy to dissolve in water, the acid group of which does not disturb the measurement. The colloidal bismuth pectin is polymeric bismuth-contained compound. The chelating between the pectin and bismuth is strong, which causes difficulty in bismuth dissolution. Measuring directly with the ultraviolet-visible spectrophotometry causes serious polymeric acid group pectin disturbance. So, the complexometric titration is adopted conventionally for the measurement of bismuth content in the colloidal bismuth pectin while the simple and accurate ultraviolet-visible spectrophotometry method is hard to use.

### SUMMARY OF THE PRESENT INVENTION

An object of the present invention is to provide a method based on the spectrophotometry for measuring the bismuth content in the colloidal bismuth pectin and the colloidal bismuth pectin-contained preparation, which is able to improve the repeatability and accuracy in measuring the bismuth content in the colloidal bismuth pectin and the colloidal bismuth pectin-contained preparation.

The method is to disperse the colloidal bismuth pectin or the colloidal bismuth pectin-contained preparation into water; add a protonic acid dissociation agent into the dispersion until a hydrogen ion concentration reaches 0.8-1.2 mol/L; centrifuge the dispersion after completely dissociation; seperate a supernatant; color the supernatant by adding a chromogen solution of citric acid or ascorbic acid and potassium iodide to make a test solution; test an absorbance of the test solution at a wavelength of 380-470 nm; compare the absorbance of the test solution with an absorbance of a reference solution of a known bismuth concentration under same conditions; calculate the bismuth content in the colloidal bismuth pectin or the colloidal bismuth pectin-contained preparation.

The protonic acid dissociation agent is nitric acid, hydrochloric acid or sulfuric acid. Optimally, the protonic acid dissociation agent is nitric acid.

The dispersion dissociated with the protonic acid is centrifuged for 5-15 minutes at a speed of 7000-10000 r/min. The dissociated polymeric pectin in the dispersion is fully settled to form a bismuth test solution without disturbance which fulfils the test requirement of the spectrophotometry.

The chromogen solution is a water solution or a 0.2-2 mol/L nitric acid solution of a potassium iodide, in which the citric acid or the ascorbic acid are added.

Furthermore, the chromogen solution comprises the citric acid or the ascorbic acid of 0.5 wt %-10 wt %, the potassium iodide of 2.5 wt %-25 wt %.

Optimally, the chromogen solution comprises the citric acid or the ascorbic acid of 2.5 wt %, the potassium iodide of 12.5 wt %.

The reference solution of the bismuth with a suitable concentration is prepared by dissolving the bismuth with the nitric acid before being diluted with water and adding the chromogen solution.

Specifically, in each 1 ml test solution or the 1 ml reference solution of the bismuth, the bismuth content is 0.1-50 μg; optimally, the in each 1 ml test solution or the 1 ml reference solution of the bismuth, the bismuth content is 2-20 μg; more optimally, in each 1 ml test solution or the 1 ml reference solution of the bismuth, the bismuth content is 5-12 μg.

A single-wavelength method is adopted for measurement of the bismuth content. A double-wavelength method is also able to be adopted to avoid disturbance.

The yellow bismuth potassium iodide generated by the bismuth and the potassium iodide has characteristic absorption spectroscopy at 399±2 nm(crest), 433±2 nm(trough), 463±2 nm(crest). When the single-wavelength method is adopted for measurement; detection wavelengths are arbitrarily chosen any one wavelength from 399 nm, 433 nm and 463 nm, wherein optimally, 463 nm is chosen.

When a double-wavelength method is adopted for measurement; detection wavelengths are arbitrarily chosen any two wavelengths from 399 nm, 433 nm and 463 nm; wherein the content is calculated with absorbance difference; optimally, the combination of 433 nm and 463 nm is chosen.

The method for measurement of bismuth content is for colloidal bismuth pectin prepared by varied methods and any colloidal bismuth pectin-contained single or compound preparation, wherein the suitable preparations comprises tablets, dispersible tablets, granules, eneric-coated tablets, colon-enteric-coated tablets, capsules, eneric- coated capsules, colon-enteric-coated capsules and dry suspensions.

The benefits of the present invention are as follow.

The present invention aims at solving the problem of quality standard for the colloidal bismuth pectin and colloidal bismuth pectin-contained preparation and uses the colloidal bismuth pectin character of forming stable colloidal system in water. First forming stable colloidal solution by adding water; then adding protonic acid into the colloidal solution until an appropriate hydrogen ion concentration is formed for the dissociation of the colloidal bismuth pectin; completely dissociating the bismuth; separating the pectin sediment completely by centrifuging; avoiding the disturbance of the pectin for the measurement of absorbance, which realizes the bismuth content measurement by the ultraviolet-visible spectrophotometry method.

Furthermore, the present invention takes the advantage of the characteristic reaction between bismuth and potassium iodide in acid medium, which forms yellow bismuth potassium iodide. Based on the reaction, the bismuth content measurement by the ultraviolet-visible spectrophotometry method is established, which is able to accurately measure the bismuth content in the colloidal bismuth pectin and the colloidal bismuth pectin-contained preparation.

The present invention adopts the diluted solution of bismuth dissolving by nitric acid as the bismuth reference solution. The purity of the bismuth is over 99.99%, Compared to mineral salts such as the bismuth nitrate, the solution is more suitable to be the reference solution.

The present invention provides a method for measuring bismuth content in the colloidal bismuth pectin and the colloidal bismuth pectin-contained preparation. The method has strong specificity, high accuracy, good repeatability, good linear relationship and high sensitivity, which is able to act as a quality control method for bismuth in a colloidal bismuth pectin or a colloidal bismuth pectin-contained preparation to effectively control the product quality.

### DETAILED DESCRIPTION OF THE PREFERRED EMBODIMENT

### Embodiment 1: Colloidal Bismuth Pectin

1) Preparation of a chromogen solution: taking 5 g ascorbic acid and 25 g potassium iodide to place in a 200 ml volumetric flask; adding 100 ml of water; shaking to dissolve; adding 25 ml of 1 mol/L nitric acid solution; adding water to dilute; dripping water to meet a scale; thus, the chromogen solution containing 2.5% of ascorbic acid, 12.5% of potassium iodide is prepared.

2) Preparation of a reference solution of bismuth: taking 275 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 2 ml of standard bismuth stock solution to place in a 100 ml volumetric flask; diluting with a nitric acid solution of 1 mol/L until a scale is reached; thus, a solution containing about 55 μg bismuth in each 1 ml is prepared as a standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 37 mg colloidal bismuth pectin powder to place in a 100 ml volumetric flask; adding 50 ml of water; shaking or impacting with an ultrasonic treatment to make the solution dissolve evenly; diluting with water until a scale is reached; thus, a colloidal solution containing about 55 μg bismuth in each 1 ml is prepared as a test stock solution; accurately weighing 5 ml of the test stock solution to place in a 15 ml centrifugal tube; accurately adding 5 ml of 2 mol/L nitric acid solution; shaking for 5 minutes; centrifuging for 10 minutes at 8000 r/min; accurately measuring 5 ml supernatant to place in a 25 ml volumetric flask; diluting with chromogen solution until a scale is reached; thus, the test solution is prepared.

4) Preparation of a blank solution: accurately measuring 5 ml of water to place in a 15 ml centrifugal tube; accurately adding 5 ml of 2 mol/L nitric acid solution; shaking for 5 minutes; accurately measuring 5 ml of the solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying a ultraviolet-visible spectrophotometry, comprising steps of using 1 cm quartz cuvette; measuring the absorbance at 463 nm wavelength; calculating a colloidal bismuth pectin content with an external standard method; a result is 14.9% (take bismuth for calculation).

### Embodiment 2: Colloidal Bismuth Pectin Capsule (Specification: 40 Mg, Take the Bismuth for Calculation)

1) Preparation of a chromogen solution: taking 2.5 g ascorbic acid and 12.5 g potassium iodide to place in a volumetric flask of 200 ml; adding 100 ml of water; shaking to dissolve; adding 25 ml of 1 mol/L nitric acid solution; adding water to dilute; dripping water to meet the scale; thus, the chromogen solution containing 1.25% of the ascorbic acid, 6.25% of the potassium iodide is prepared.

2) Preparation of a reference solution of bismuth: taking 250 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 1 ml of the standard bismuth stock solution to place in a 10 ml volumetric flask; diluting with a nitric acid solution of 1.2 mol/L until a scale is reached; thus, a solution containing about 250 μg bismuth in each 1 ml is prepared as a standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 510 mg content in a colloidal bismuth pectin capsule to place in a 100 ml volumetric flask; adding 50 ml of water; shaking or impacting with an ultrasonic treatment to make the solution dissolve evenly; diluting with water until a scale is reached; thus, a dispersion containing about 500 μg bismuth in each 1 ml is prepared as a test stock solution; accurately weighing 5 ml of test stock solution to place in a 15 ml centrifugal tube; accurately adding 5 ml of 2.4 mol/L nitric acid solution; shaking for 5 minutes; centrifuging for 15 minutes at 7000 r/min; accurately measuring 5 ml supernatant to place in a 25 ml volumetric flask; diluting with the chromogen solution until a scale is reached; thus, the test solution is prepared.

4) Preparation of a blank solution: accurately measuring 5 ml of water to place in a 15 ml centrifugal tube; accurately adding 5 ml of 2.4 mol/L nitric acid solution; shaking for 5 minutes; accurately measuring 5 ml of a solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying a ultraviolet-visible spectrophotometry, comprising steps of using 1 cm quartz cuvette; measuring an absorbance at 433 nm wavelength; calculating a colloidal bismuth pectin content with an external standard method; a result is 100.2% of the labeled amount(take bismuth for calculation).

### Embodiment 3: Colloidal Bismuth Pectin Capsule (Specification: 50 Mg, Take the Bismuth for Calculation)

1) Preparation of a chromogen solution: taking 10 g ascorbic acid and 50 g potassium iodide to place in a volumetric flask of 200 ml; adding 100 ml of water; shaking to dissolve; adding 25 ml of 1 mol/L nitric acid solution; adding water to dilute; dripping water to meet the scale; thus, the chromogen solution containing 5% of an ascorbic acid, 25% of a potassium iodide is prepared.

2) Preparation of a reference solution of bismuth: taking 500 mg accurately weighed bismuth to place in a 100 ml volumetric flask; adding 6.4 ml nitric acid to dissolve the bismuth; diluting with water until the scale is reached; thus, a standard bismuth stock solution is prepared; accurately weighing 1 ml of standard bismuth stock solution to place in a 100 ml volumetric flask; diluting with a hydrochloric acid solution of 0.8mol/L until the scale is reached; thus, a solution containing about 50 μg bismuth in each 1 ml is prepared as the standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 110 mg content in a colloidal bismuth pectin capsule to place in a 100 ml volumetric flask; adding 50 ml of water; shaking or impacting with an ultrasonic treatment to make a solution dissolve evenly; diluting with water until a scale is reached; thus, a dispersion containing about 100 μg bismuth in each 1 ml is prepared as a test stock solution; accurately weighing 5 ml of the test stock solution to place in a 15 ml centrifugal tube; accurately adding 5 ml of 1.6 mol/L hydrochloric acid solution; shaking for 5 minutes; centrifuging for 10 minutes at 7000 r/min; accurately measuring 5 ml supernatant to place in a 25 ml volumetric flask; diluting with chromogen solution until a scale is reached; thus, the test solution is prepared.

4) Preparation of a blank solution: accurately measuring 5 ml of water to place in a 15 ml centrifugal tube; accurately adding 5 ml hydrochloric acid solution of 1.6 mol/L; shaking for 5 minutes; accurately measuring 5 ml solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying a ultraviolet-visible spectrophotometry, comprising steps of using 1 cm quartz cuvette; measuring an absorbance at 463 nm wavelength; calculating a colloidal bismuth pectin content with an external standard method; a result is 98.4% of a labeled amount(take bismuth for calculation).

### Embodiment 4: Colloidal Bismuth Pectin Capsule (Specification: 100 Mg, Take the Bismuth for Calculation)

1) Preparation of a chromogen solution: taking 2 g citric acid and 20 g potassium iodide to place in a volumetric flask of 200 ml; adding 100 ml of water; shaking to dissolve; adding water to dilute; dripping water to meet a scale; thus, the chromogen solution containing 1% of a citric acid, 10% of a potassium iodide is prepared.

2) Preparation of a reference solution of bismuth: taking 275 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 1.5 ml of standard bismuth stock solution to place in a 100 ml volumetric flask; diluting with a sulfuric acid solution of 0.5 mol/L until a scale is reached; thus, a solution containing about 41.25 μg bismuth in each 1 ml is prepared as a standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 63.7 mg content in a colloidal bismuth pectin capsule to place in a 100 ml volumetric flask; adding 50 ml of water; shaking or impacting with an ultrasonic treatment to make the solution dissolve evenly; diluting with water until a scale is reached; thus, a dispersion containing about 82.5 μg bismuth in each 1 ml is prepared as a test stock solution; accurately weighing 5 ml of test stock solution to place in a 15 ml centrifugal tube; accurately adding 5 ml of 1 mol/L sulfuric acid solution; shaking for 5 minutes; centrifuging for 10 minutes at 10000 r/min; accurately measuring 5 ml of supernatant to place in a 25 ml volumetric flask; diluting with chromogen solution until a scale is reached; thus, the test solution is prepared.

4) Preparation of a blank solution: accurately measuring 5 ml of water to place in a 15 ml centrifugal tube; accurately adding 5 ml of 1 mol/L sulfuric acid solution; shaking for 5 minutes; accurately measuring 5 ml of a solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying an ultraviolet-visible spectrophotometry, comprising steps of using 1 cm quartz cuvette; measuring an absorbance at 433 nm and 463 nm wavelength; calculating a colloidal bismuth pectin content with an external standard method according to an absorbance difference at the two wavelength; a result is 99.6% of a labeled amount bismuth for calculation).

### Embodiment 5: Colloidal Bismuth Pectin Dispersible Tablet (Specification: 50 Mg, Take the Bismuth for Calculation)

1) Preparation of a chromogen solution: taking 20 g citric acid and 50 g potassium iodide to place in a volumetric flask of 200 ml; adding 100 ml of water; shaking to dissolve; adding 25 ml of 5 mol/L nitric acid solution; dripping water to meet a scale; thus, the chromogen solution containing 10% of a citric acid, 25% of a potassium iodide is prepared.

2) Preparation of a reference solution of bismuth: taking 275 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 0.4 ml of standard bismuth stock solution to place in a 100 ml volumetric flask; diluting with a nitric acid solution of 0.9 mol/L until a scale is reached; thus, a solution containing about 11 μg bismuth in each 1 ml is prepared as a standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: taking 20 colloidal bismuth pectin dispersible tablets to finely grind; accurately weighing 33.8 mg of the powder to place in a 100 ml volumetric flask; adding 50 ml of water; shaking or impacting with an ultrasonic treatment to make a solution dissolve evenly; diluting with water until a scale is reached; thus, a dispersion containing about 22 μg bismuth in each 1 ml is prepared as a test stock solution; accurately weighing 5 ml test stock solution to place in a 15 ml centrifugal tube; accurately adding 5 ml of 1.8 mol/L nitric acid solution; shaking for 5 minutes; centrifuging for 10 minutes at 8000 r/min; accurately measuring 5 ml of supernatant to place in a 25 ml volumetric flask; diluting with the chromogen solution until a scale is reached; thus, the test solution is prepared.

4) Preparation of a blank solution: accurately measuring 5 ml of water to place in a 15 ml centrifugal tube; accurately adding 5 ml of 1.8 mol/L nitric acid solution; shaking for 5 minutes; accurately measuring 5 ml solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying a ultraviolet-visible spectrophotometry, comprising steps of using 1 cm quartz cuvette; measuring an absorbance at 399 nm wavelength; calculating a colloidal bismuth pectin content with an external standard method; a result is 99.0% of a labeled amount (take bismuth for calculation).

### Embodiment 6: Colloidal Bismuth Pectin Granules (Specification: 150 Mg, Take the Bismuth for Calculation)

1) Preparation of a chromogen solution: taking 8 g ascorbic acid and 30 g potassium iodide to place in a volumetric flask of 200 ml; adding 100 ml of water; shaking to dissolve; adding 25 ml of 1 mol/L nitric acid solution; adding water to dilute; dripping water to meet a scale; thus, the chromogen solution containing 4% of an ascorbic acid, 15% of a potassium iodide is prepared.

2) Preparation of a reference solution of bismuth: taking 50 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 1 ml standard bismuth stock solution to place in a 100 ml volumetric flask; diluting with a nitric acid solution of 1.1 mol/L until a scale is reached; thus, a solution containing about 5 μg bismuth in each 1 ml is prepared as the standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: taking the colloidal bismuth pectin granules to finely grind; accurately weighing 19 mg of the powder to place in a 100 ml volumetric flask; adding 50 ml of water; shaking or impacting with an ultrasonic treatment to make the solution dissolve evenly; diluting with water until a scale is reached; thus, a dispersion containing about 10 μg bismuth in each 1 ml is prepared as a test stock solution; accurately weighing 5 ml test stock solution to place in a 15 ml centrifugal tube; accurately adding 5 ml of 2.2 mol/L nitric acid solution; shaking for 5 minutes; centrifuging for 15 minutes at 9000r/min; accurately measuring 5 ml supernatant to place in a 25 ml volumetric flask; diluting with chromogen solution until a scale is reached; thus, the test solution is prepared.

4) Preparation of a blank solution: accurately measuring 5 ml of water to place in a 15 ml centrifugal tube; accurately adding 5 ml of 2.2mol/L nitric acid solution; shaking for 5 minutes; accurately measuring 5 ml of a solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying an ultraviolet-visible spectrophotometry, comprising steps of using 1 cm quartz cuvette; measuring an absorbance at 463 nm wavelength; calculating a colloidal bismuth pectin content with an external standard method; the result is 99.0% of a labeled amount(take bismuth for calculation).

### Embodiment 7: Compound Colloidal Bismuth Pectin Capsule (Comprising Colloidal Bismuth Pectin, Metronidazole and Tetracycline Hydrochloride; Every Capsule Containing 35 Mg Colloidal Bismuth Pectin which is Calculated with Bismuth)

1) Preparation of a chromogen solution: taking 15 g ascorbic acid and 40 g potassium iodide to place in a volumetric flask of 200ml; adding 100 ml of water; shaking to dissolve; adding 25 ml of 1 mol/L nitric acid solution; adding water to dilute; dripping water to meet a scale; thus, the chromogen solution containing 7.5% of an ascorbic acid, 20% of a potassium iodide is prepared.

2) Preparation of a reference solution of bismuth: taking 275 mg accurately weighed bismuth to place in a 100 ml volumetric flask; adding 6.4 ml nitric acid to dissolve the bismuth; diluting with water until a scale is reached; thus, the standard bismuth stock solution is prepared; accurately weighing 1 ml of standard bismuth stock solution to place in a 100 ml volumetric flask; diluting with a nitric acid solution of 0.8 mol/L until a scale is reached; thus, a solution containing about 27.5 μg bismuth in each 1 ml is prepared as the standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 184 mg content of the compound colloidal bismuth pectin dispersible capsules to place in a 100 ml volumetric flask; adding 50 ml of water; shaking or impacting with an ultrasonic treatment to make the solution dissolve evenly; diluting with water until a scale is reached; thus, a dispersion containing about 55 μg bismuth in each 1 ml is prepared as the test stock solution; accurately weighing 5 ml of test stock solution to place in a 15 ml centrifugal tube; accurately adding 5 ml of 1.6 mol/L nitric acid solution; shaking for 5 minutes; centrifuging for 5 minutes at 10000 r/min; accurately measuring 5 ml of supernatant to place in a 25 ml volumetric flask; diluting with the chromogen solution until a scale is reached; thus, the test solution is prepared.

4) Preparation of a blank solution: accurately measuring 5 ml of water to place in a 15 ml centrifugal tube; accurately adding 5 ml of 1.6 mol/L nitric acid solution; shaking for 5 minutes; accurately measuring 5 ml solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying a ultraviolet-visible spectrophotometry, comprising steps of using 1 cm quartz cuvette; measuring an absorbance at 399 nm and 463 nm wavelength; calculating the colloidal bismuth pectin content with an external standard method according to the absorbance difference at the two wavelength; a result is 101.3% of a labeled amount(take bismuth for calculation).

### Embodiment 8: Colloidal Bismuth Pectin Powder (Specification: 150 Mg, Take the Bismuth for Calculation)

1) Preparation of a chromogen solution: taking 1 g ascorbic acid and 5 g potassium iodide to place in a volumetric flask of 200 ml; adding 100 ml of water; shaking to dissolve; adding 25 ml of 1 mol/L nitric acid solution; adding water to dilute; dripping water to meet a scale; thus, the chromogen solution containing 0.5% of ascorbic acid, 2.5% of potassium iodide is prepared.

2) Preparation of a reference solution of bismuth: taking 10 mg accurately weighed bismuth to place in a 100 ml volumetric flask; adding 6.4 ml nitric acid to dissolve the bismuth; diluting with water until a scale is reached; thus, a standard bismuth stock solution is prepared; accurately weighing 1 ml of standard bismuth stock solution to place in a 100 ml volumetric flask; diluting with a hydrochloric acid solution of 1 mol/L until a scale is reached; thus, a solution containing about 1 μg bismuth in each 1 ml is prepared as a standard bismuth solution; accurately weighing 5 ml standard bismuth solution to place in a 25 ml volumetric flask; diluting with the chromogen solution until a scale is reached; thus, the reference solution of bismuth is prepared.

3) Preparation of a test solution: accurately weighing 10 mg of the colloidal bismuth pectin powder to place in a 500 ml volumetric flask; adding 50 ml of water; shaking or impacting with an ultrasonic treatment to make the solution dissolve evenly; diluting with water until a scale is reached; thus, a dispersion containing about 2 μg bismuth in each 1 ml is prepared as the test stock solution; accurately weighing 5 ml test stock solution to place in a 15 ml centrifugal tube; accurately adding 5 ml of 1.9 mol/L hydrochloric acid solution; shaking for 5 minutes; centrifuging for 10 minutes at 8000 r/min; accurately measuring 5 ml supernatant to place in a 25 ml volumetric flask; diluting with chromogen solution until a scale is reached; thus, the test solution is prepared.

4) Preparation of a blank solution: accurately measuring 5 ml of water to place in a 15 ml centrifugal tube; accurately adding 5 ml 1.9 mol/L hydrochloric acid solution; shaking for 5 minutes; accurately measuring 5 ml of the solution to place in a 25 ml volumetric flask; diluting with the chromogen solution until a scale is reached; thus the blank solution is prepared.

5) Measuring: taking the reference solution and the test solution of the bismuth; taking the blank solution as a reference; applying an ultraviolet-visible spectrophotometry, comprising steps of using 1 cm quartz cuvette; measuring an absorbance at 463 nm wavelength; calculating the colloidal bismuth pectin content with an external standard method; a result is 99.0% of a labeled amount(take bismuth Bi for calculation).

### Embodiment 9: Test for Accuracy

Taking the bismuth reference solution; testing the absorbance at 463 nm wavelength; repeating the test for 6 times, wherein the results are 0.6494, 0.6494, 0.6495, 0.6494, 0.6494, 0.6495 respectively; the RSD (relative standard deviation) is 0.1%; the accuracy of the device is good.

### Embodiment 10: Linear Range

Preparing a series of standard bismuth solution concentrations of which are 87.5 μg/ml, 55 μg/ml, 27.5 μg/ml, 11 μg/ml, 8.8μg/ml, 5.5 μg/ml, 2.2 μg/ml, 1.1 μg/ml, 0.55 μg/ ml respectively; accurately weighing 5 ml of each of the series of standard bismuth solution to place in a 25 ml volumetric flask; diluting with the chromogen solution until a scale is reached; measuring an absorbance at the 463 nm wavelength; using the least square to linearly regress an absorbance and the concentration of the standard bismuth solution; a regression formula is A=0.0114C+0.0032; a relative coefficient is R²=0.9999; the standard bismuth solution shows good linear relationship within a concentration range of 0.55-87.5 μg/mL.

### Embodiment 11: Limit of Detection and Limit of Quantification

Preparing 20 blank solutions and measuring an absorbance at 463 nm wavelength, wherein absorbance are 0.0434, 0.0449, 0.0441, 0.0451, 0.0436, 0.0424, 0.0438, 0.0434, 0.0451, 0.0445, 0.0444, 0.0445, 0.0443, 0.0455, 0.0441, 0.0446, 0.0444, 0.0443, 0.0441 and 0.0445 respectively; a calculation standard deviation SD equals 0.00070; according to the regulation of International Union of Pure and Applied Chemistry (IUPAC) on the limit of detection, a calculated limit of detection=3×0.00070=0.0021 which is equivalent to a standard bismuth solution of 0.15 μg/ml; a limit of quantification=10×0.00070=0.0070 which is equivalent to a standard bismuth solution of 0.5 μg/ml.

### Embodiment 12: Recovery Rate

Taking proper amount of the content in a colloidal bismuth pectin capsule (equivalent to 2.75 mg bismuth) to place in 100 ml volumetric flasks; adding 0.8 ml, 1 ml, 1.2 ml of the standard bismuth stock solution (2.75 mg/ml) respectively; adding 50 ml water and impacting with ultrasonic treatment to make the solution evenly dispersed; diluting with water until a scale is reached; thus, the stock solution is prepared.

Taking 5 ml stock solution to place in a 10 ml centrifugal tube; accurately adding 5 ml of 2 mol/L nitric acid solution; shaking and centrifuging; accurately measuring 5 ml of supernatant to place in a 25 ml volumetric flask; diluting with the chromogen solution until a scale is reached; repeatedly preparing 3 solutions for each stock solutions and measuring an absorbance at 463 nm wavelength; a calculated recovery rates are 99.28%, 100.69%, 99.77%, 97.93%, 101.57%, 99.16%, 99.40%, 100.86% and 99.64% respectively; an average recovery rate is 99.81% and RSD=1.09%, which shows the method has good recovery rate and accuracy.

### Embodiment 13: Repeatability

Preparing 6 bismuth reference solutions and test solutions respectively and measuring an absorbance; absorbance of reference solutions are 0.6537, 0.6479, 0.6487, 0.6509, 0.6539 and 0.6531 respectively, RSD=0.4%; contents of the test article are 98.40%, 98.74%, 98.66%, 98.35%, 98.55% and 98.62% of a labeled amount respectively, RSD=0.2%; the method has good repeatability.

### Embodiment 14: The Stability of the Solution

Taking the test solution and measuring an absorbance in 0, 2, 4, 6, 8 hours, wherein absorbance are 0.6528, 0.6558, 0.6489, 0.6514, 0.6504 and 0.6538 respectively, RSD=0.4%; the test solution has good stability within 8 hours.

Accurately weighing the proper amount of the content in a colloidal bismuth pectin capsule (about 5.5 mg bismuth) to place in a 100 ml volumetric flask; adding 50 ml of water; impacting with an ultrasonic treatment to disperse the solution evenly; diluting with water until a scale is reached; taking 5 ml of the colloidal solution in 0, 2, 4, 6, 8 hours to place in a 10 ml centrifugal tube; accurately adding 5 ml of 2mol/L nitric acid solution; shaking and centrifuging for 10 minutes at 8000 r/min; accurately taking 5 ml supernatant to place in a 25 ml volumetric flask; diluting with a chromogen solution until a scale is reached; measuring absorbance and calculating bismuth contents which are 98.79%, 98.27%, 98.55%, 98.19%, 98.66% and 98.82% of a labeled amount respectively, RSD=0.3%; a stability of a colloidal solution is good within 8 hours and the colloidal solution is able to keep in a homogeneous state.

### Embodiment 15: contrast test for ultraviolet-visible spectrophotometry and complexometric titration

1. Complexometric Titration

Accurately weighing 0.5 g colloidal bismuth pectin, adding 5 ml nitric acid solution (1→2); dissolving a solution by heating; adding 150 ml of water and two drops of xylenol orange indicator; titrating the solution with EDTA-2Na (ethylenediaminetetraacetic acid disodium salt dehydrate) volumetric solution of 0.05 mol/L until the solution appears yellow; each 1 ml EDTA-2Na volumetric solution is equivalent to 10.45 mg bismuth(Bi); testing in parallel for 6 times, wherein results are 15.26%, 14.87%, 15.14%, 14.72%, 14.69% and 14.93% respectively, averaged 14.94%, RSD%=1.52%.

Accurately weighing 0.25 g colloidal bismuth pectin; adding 5 ml nitric acid solution (1→2); dissolving a solution by heating; adding 1.2 ml, 1.5 ml, 1.8 ml of standard bismuth stock solutions (27.5 mg/ml); adding 150 ml of water and two drops of xylenol orange indicator; titrating the solution with EDTA-2Na volumetric solution of 0.05 mol/L until the solution appears yellow; each 1 ml EDTA-2Na volumetric solution is equivalent to 10.45 mg bismuth Bi; repeatedly preparing 3 of each of the solution for test; calculated recovery rates are 102.95%, 98.13%, 101.64%, 103.76%, 98.07%, 99.12%, 101.69%, 103.26% and 102.31% respectively, an averaged recovery rate is 101.21%, RSD=2.18%.

2. Ultraviolet-visible spectrophotometry.

Accurately weighing 37 mg colloidal bismuth pectin; testing in parallel according to the embodiment 1 for 6 times, wherein the results are 14.86%, 14.95%, 14.90%, 14.88%, 14.99% and 14.83% respectively, averaged 14.90%, RSD%=0.40%.

Taking 37 mg colloidal bismuth pectin to place in 100 ml volumetric flasks; adding 1.6 ml, 2.0 ml and 2.4 ml standard bismuth stock solution (2.75 mg/ml) respectively; adding 50 ml of water and impacting with the ultrasonic treatment to disperse the solution evenly; diluting with water until a scale is reached; thus, the stock solution is prepared; taking 5 ml of the stock solution to place in a 10 ml centrifugal tube; accurately adding 5 ml nitric acid solution of 2 mol/L; shaking and centrifuging; accurately measuring 5 ml supernatant to place in a 25 ml volumetric flask; diluting with the chromogen solution until a scale is reached; repeatedly prepare 3 parts of each stock solution; measuring an absorbance at 463 nm wavelength; calculated recovery rates are 98.89%, 101.38%, 99.86%, 101.71%, 100.87%, 99.09%, 101.25%, 100.91% and 100.71% respectively; an average recovery rate is 100.52%, RSD=1.00%.

The present invention solves the difficulty in complexometric titration of end point detection. The recovery rate, repeatability and other features of the measurement are superior to the complexometric titration.

What is claimed is:

1. A method for measuring a bismuth content in a colloidal bismuth pectin or a colloidal bismuth pectin-contained preparation, comprising steps of: dispersing the colloidal bismuth pectin or the colloidal bismuth pectin-contained preparation into water;
adding a protonic acid dissociation agent into a dispersion until a hydrogen ion concentration reaches 0.8-1.2 mol/L; centrifuging the dispersion after completely dissociation; seperating a supernatant; coloring the supernatant by adding a chromogen solution of a citric acid or an ascorbic acid and a potassium iodide to form a test solution;
testing an absorbance of the test solution at a wavelength of 380-470 nm; comparing the absorbance of the test solution with an absorbance of a reference solution of a known bismuth concentration under same conditions; calculating the bismuth content in the colloidal bismuth pectin or the colloidal bismuth pectin-contained preparation.

2. The method as recited in claim 1, wherein the protonic acid dissociation agent is a nitric acid, a hydrochloric acid or a sulfuric acid.

3. The method as recited in claim 2, wherein the protonic acid dissociation agent is the nitric acid.

4. The method as recited in claim 1, wherein the chromogen solution is a water solution or a 0.2-2 mol/L nitric acid solution of the citric acid or the ascorbic acid and the potassium iodide.

5. The method as recited in claim 4, wherein the chromogen solution comprises the citric acid or the ascorbic acid of 0.5 wt %- 10 wt %, the potassium iodide of 2.5 wt %-25 wt %.

6. The method as recited in claim 4, wherein the chromogen solution comprises the citric acid or the ascorbic acid of 2.5 wt %, the potassium iodide of 12.5 wt %.

7. The method as recited in claim 1, wherein the reference solution of the bismuth is prepared by dissolving the bismuth with the nitric acid before being diluted with water and adding the chromogen solution.

8. The method as recited in claim 1, wherein in each 1 ml test solution or the 1 ml reference solution of the bismuth, a bismuth content is 0.1-50 m.

9. The method as recited in claim 1, wherein in each 1 ml test solution or the 1 ml reference solution of the bismuth, the bismuth content is 2-20 μg.

10. The method as recited in claim 1, wherein in each 1 ml test solution or the 1 ml reference solution of the bismuth, the bismuth content is 5-12 μg.

11. The method as recited in claim 1, wherein the dispersion dissociated with the protonic acid is centrifuged for 5-15 minutes at a speed of 7000-10000 r/min.

12. The method as recited in claim 1, wherein a single-wavelength method is adopted for measurement; detection wavelengths are arbitrarily chosen any one wavelength from 399 nm, 433 nm and 463 nm.

13. The method as recited in claim 12, the detection wavelength is 463 nm.

14. The method as recited in claim 1, wherein a double-wavelength method is adopted for measurement; detection wavelengths are arbitrarily chosen any two wavelengths from 399 nm, 433 nm and 463 nm.

15. The method as recited in claim 14, the detection wavelengths are 433 nm and 463 nm.

16. The method as recited in claim 1, wherein the colloidal bismuth pectin-contained preparation is a single preparation or a compound preparation comprises tablets, dispersible tablets, eneric-coated tablets, colon-enteric-coated tablets, capsules, soft capsules, eneric-coated capsules, colon-enteric-coated capsules, granules, dripping pills, microcapsules and dry suspensions.
